(19) United States
(12) Patent Application Publication
(10) Pub. No.: US 2004/0048827 A1
(43) Pub. Date: Mar. 11, 2004

(54) S-adenosylmethionine and derivatives thereof for the treatment and prevention of alzheimer's disease

(76) Inventors: Sigfrido Scarpa, Rome (IT); Andrea Fuso, Pomezia (RM) (IT)

Correspondence Address: Arent Fox Kintner Plotkin & Kahn, 1050 Connecticut Avenue NW, Suite 400, Washington, DC, 20036-5339, US

(21) Appl. No.: 10/398,748

(22) PCT Filed: Oct. 18, 2001
(86) PCT No.: PCT/IT01/00528

(30) Foreign Application Priority Data
Oct. 20, 2000 (IT) RM2000A000556

(51) Int. Cl. A61K 31/7076

(52) U.S. Cl. 514/46

(57) ABSTRACT

Use of S-adenosylmethionine and derivatives thereof for the preparation of a medicament for the treatment and prevention of Alzheimer's disease and regulation of the expression of genes as β-secretase, presenilin-1, presenilin-2 γ-amyloid protein precursor.

The present invention relates to the use of S-adenosylmethionine (SAM) and derivatives thereof for the preparation of a medicament for the treatment and prevention of Alzheimer's disease.

Alzheimer's disease, so named as described for the first time in 1906 by Alois Alzheimer, German neuropathologist, is diffusing rapidly due to the human life lengthening. It is a form of late age dementia which is caused by neuron degeneration in encephalon large areas: cerebral cortex, amygdala, hippocampus. It is also known, for a limited number of cases (about 5% of the total Alzheimer patients), a type of Alzheimer's disease characterised by inheritance, early onset (40-60 year age), rapid evolution (within 2-3 years).

The disease shows two main characteristics from the molecular point of view: the formation of dense plaques insoluble within the intercellular spaces of β-amyloid protein (amyloid plaques) and so named “neurofibrillar tangles”, within the neurons, resulting from the modification of Tau protein, which is a protein necessary for the microfilament assembling. It is now verified that the formation and build-up of the amyloid plaque, anyway present in “normal” aged humans in much smaller amounts, result in neuronal degeneration typical for the Alzheimer patient through a presently unknown pattern.

The formation of amyloid plaque depends on a group of proteins involved in the processing of the precursor of the β-amyloid protein (APP), a 695 amino acid protein whose function is up to now unknown: α-secretase, presenilin-1 (PS1), γ-secretase, presenilin-2, β-secretase (1). In the synthesis process of Aβ amyloid peptide the amyloid protein precursor (APP) is cleaved by β-secretase which releases its extracellular domain. Presenilin is processed following its synthesis by an unknown protease, named presenilinase, resulting in N-terminal and C-terminal fragments. These fragments remain not covalently bonded forming active γ-secretase. Each fragment contains an aspartyl residue (Asp 257 or -Asp 385) in the active site. The β-secretase cleaved APP fragment is bonded to presenilin and is cleaved by the latter in an unidentified subcellular compartment. Aβ is released into the extracellular environment and associates to amyloid plaques identified in the brain of Alzheime's disease affected patients.

It is known that α-secretase cleaves the β-amyloid protein precursor resulting in polypeptides which are rapidly degraded, while on the contrary γ- and β-secretase produce 1-40 and 1-42 amyloidogenic polypeptides which self-associate forming the amyloid plaque. Presenilin-1 (PS1), which seems to be identified as γ-secretase, and presenilin-2 (PS2) are directly involved in the γ- and β-secretase cleavage. By PS1 and PS2 gene “knock-out” experiments (gene inactivation) it is observed that the 1-40 and 1-42 β-amyloid (Aβ) production is suppressed and their complete elimination leads to the cell death.

It is known that in the early onset familial Alzheimr's disease the production of Aβ peptide is affected by PS1 and PS2 gene mutation.

The interest towards the presenilins has increased by the evidence that PS1-deficient mouse embryos generated neuronal cultures wherein PS1 expression and γ-secretase activity were both absent (2) and that γ-secretase activity was inhibited by direct mutagenesis of either of the two aspartyl moieties in transmembrane domains 6 and 7 of PS1 (3). Furthermore the paper of Li et al. (4) has contributed to come to the conclusion that γ-secretase is PS1 itself. The observations made by Brown et al. (5) about the physiological role of presenilins as regulators is also sustained by the discovery that PS1 deficiency in mice was lethal (6,7). All these indications suggest that PS1 may be the target for the therapy in Alzheimer's disease, although its expression could not be completely blocked.

In the last years the studies have been focused on the research of a medicament or method for the reduction of the presenilin-1 and therefore β-amyloid synthesis. A substance recently described by Li et al. (4) as blocking presenilin-1 protein does not seem to be suitable as therapeutic agent because it is unable to go beyond the blood-brain barrier and eliminates completely the β-amyloid protein. Furthermore recently a 1-40 and 1-42 β-amyloid vaccine was tested, however again it is non suitable for the therapy because it eliminates completely also 1-40 β-amyloid, which in small doses exerts physiological functions.

No method up to now was tested to reduce the expression of the genes which cause a build-up of β-amyloid protein.

Accordingly to the above reported therefore it results in the need to provide an effective product for the prevention and treatment of Alzheimer's disease suitable to overcome the above described problems.

It is known that the regulation of gene expression occurs by DNA methylation on the promoter and many genes are expressed when some promoter sites are de-methylated.

It was observed that in the brain of Alzheimer patients, post mortem, the concentration of S-adenosylmethionine (SAM), which is the major methyl donor in living organisms, is much lower than normal (8, 9). Therefore it is likely that Alzheimer's disease can be related to the decreasing of DNA methylation which would result in over-expression of some genes involved in the processing of the β-amyloid protein precursor (APP) at the expense of others. Such a dysfunction would result in a build-up of Aβpeptide within the senile plaques.

PS1 has been shown to be involved in the cleavage of Notch-1, a signal transduction protein, in the neuronal differentiation (10) and probably other physiological functions. However PS1 or γ-secretase over-expression at the expense of α-secretase could cause a build-up of 1-40 and 1-42 peptides which, over the years, would lead to the disease. Therefore, although the PS1 blockage aimed at reducing the formation of β-amyloid is a successful strategy, a drastic blockage that causes Notch-1 no-activation has to be avoided, wherein Notch-1 is a fundamental factor also for the maturation of stem cells, particularly for those of ematopoietic line.

As above mentioned it is known that the activation of the gene expressions occurs when cytosine residues in the CpG and non CpG portions are de-methylated (11-13), however, up to now, there are no reports on the gene silencing by the administration of the methyl donor (SAM).

The applicant of the invention, in his research efforts, surprisingly found that the administration of S-adenosylmethionine, in a form suitable to go beyond the cell membrane, increases the endocellular levels of this substance resulting in methylation of at least one site regulating the expression of the PS1 gene, a β-amyloid protein precursor, thus repressing this expression without a complete blocking.

Particularly the SAM administration in cellular cultures of human neuroblastoma showed a PS1 remarkable decrease and the increase block of PS2, β-secretase and APP expression restoring the metabolic equilibrium in favour of α-secretase.

It is therefore an object of the present invention the use of S-adenosylmethionine and derivatives thereof for the preparation of a medicament for the treatment and prevention of Alzheimer's disease

According to a preferred embodiment thereof as S-adenosylmethionine derivative the di-sulfonate form is used because more water soluble and suitable to go beyond the blood-brain barrier.

It is a further object of the invention the use of S-adenosylmethionine and derivatives thereof for the preparation of a medicament for the regulation of the expression of genes belonging to the group of β-secretase, presenilin-1, presenilin-2, β-amyloid protein precursor. Particularly the regulation can occur by means of the modulation of at least one methylation site of gene regulating regions.

The medicaments comprising S-adenosylmethionine and/or derivatives thereof and one or more pharmaceutically acceptable carriers can be administrated both by oral and parenteral route. The formulations can be prepared in solid forms as, for example, tablets and capsules or in liquid forms as, for example, injectable solutions, syrups, emulsions.

The present invention will be now described, by way of illustration, but not limitation, according to preferred embodiments thereof, particularly referring to the enclosed drawings, wherein:

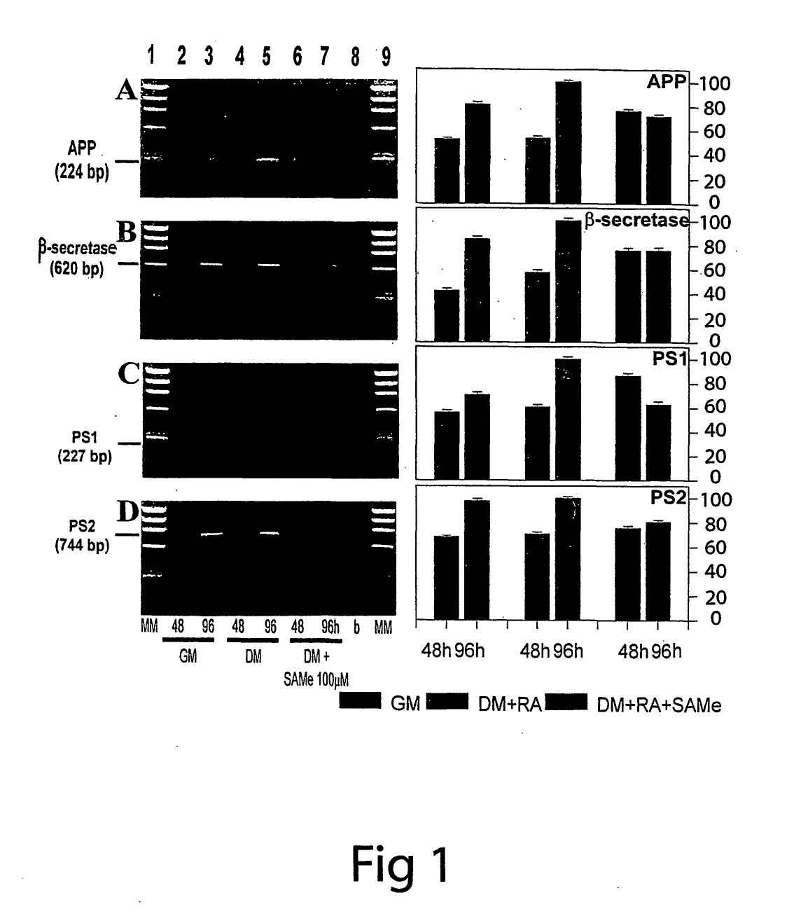

FIG. 1 shows the expression of genes involved within Alzheimer's disease: APP (A): β-secretase (B), PS1 (C) and PS2 (D) after 48 or 96 hours of culture, obtained by “Northern blot” technique. SK-N-SH human neuroblastoma cells were grown, respectively, in a growth medium (GM) containing 8% foetal calf serum (FCS) (2-3 lanes), in a differentiation medium (DM) containing 1% foetal calf serum (FCS) and retinoic acid (RA) (lanes 4-5) and in DM medium in the presence of 100 μM SAM (lanes 6-7). On the right the plots of the optical density (O.D.) values, obtained from electrophoresis signals, normalised to γ-actin (not shown) and expressed as percent average are showed.

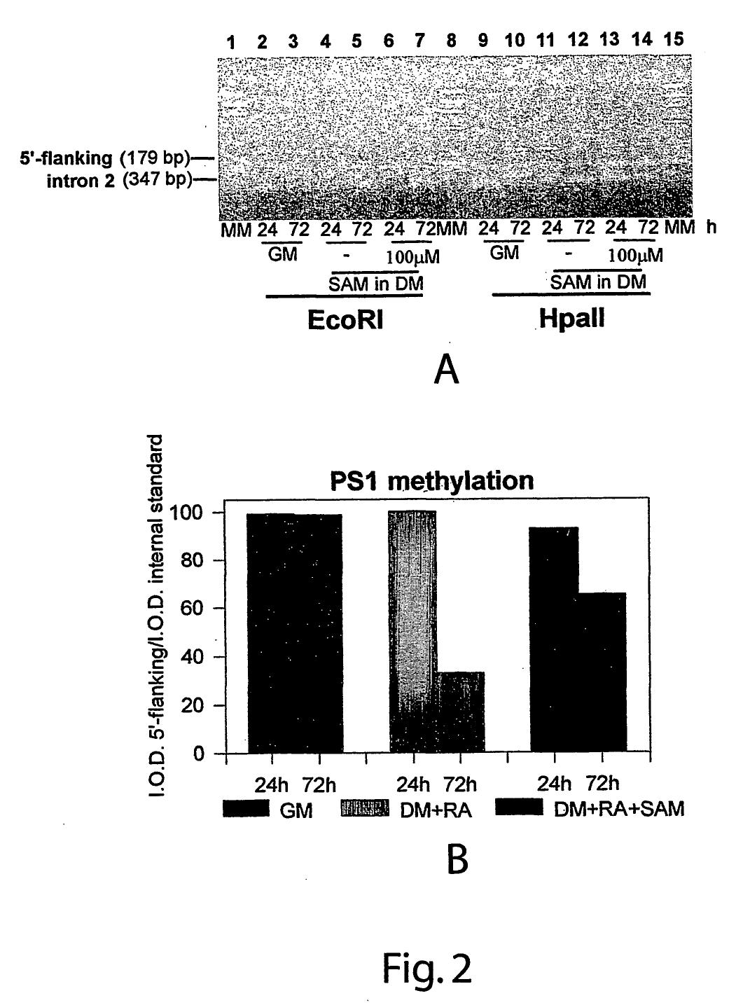

FIG. 2A shows an electrophoresis run of DNA fragments from the PS1 gene promoter region after PCR, EcoRi (left) or Hpall (right) digestion. Lanes 2, 9: GM, 24 hours; lanes 3, 10: GM, 72 hours; lanes 4, 11: DM, 24 hours; lanes 5,12: DM, 72 hours; lanes 6, 13: SAM in DM, 24 hours; lanes 7, 14: SAM in DM, 72 hours; lanes 1, 8, 15 molecular weight markers. B panel represents a densitometry histogram of the panel A electrophoresis bands.

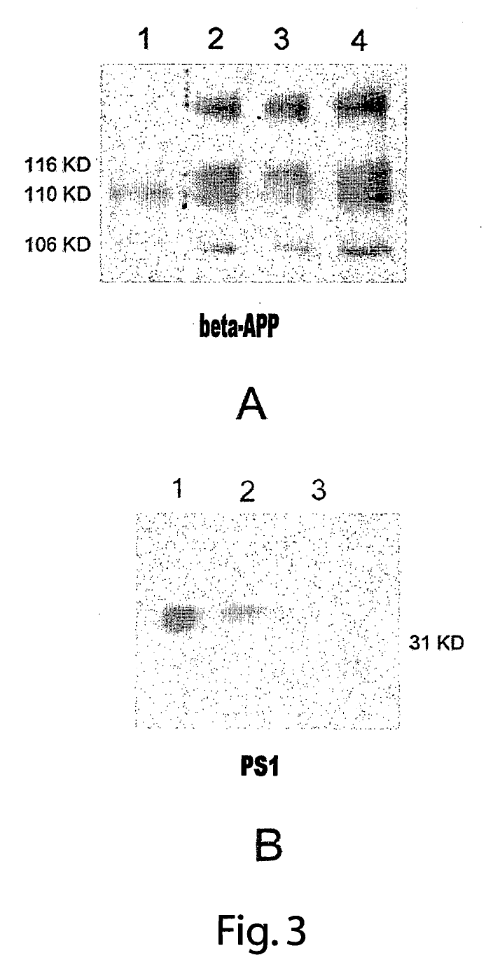

FIG. 3A shows the levels of the amyloid protein precursor in extracts of the cells grown respectively in GM (lane-1), DM (lane 2), DM in the presence of 10 μM retinoic acid (RA) (lane 3) and DM in the presence of 10 μM retinoic acid (RA) and SAM 100 μM (lane 4) after 120 hours of culture. Panel B shows the levels of presenilin-1 in the extracts of cells grown respectively in GM (lane 1), DM in the presence of 10 μM retinoic acid (RA) (lane 2) and DM in the presence of 100 μM SAM 100 and retinoic acid (RA) (lane 3) after 120 hours of culture.

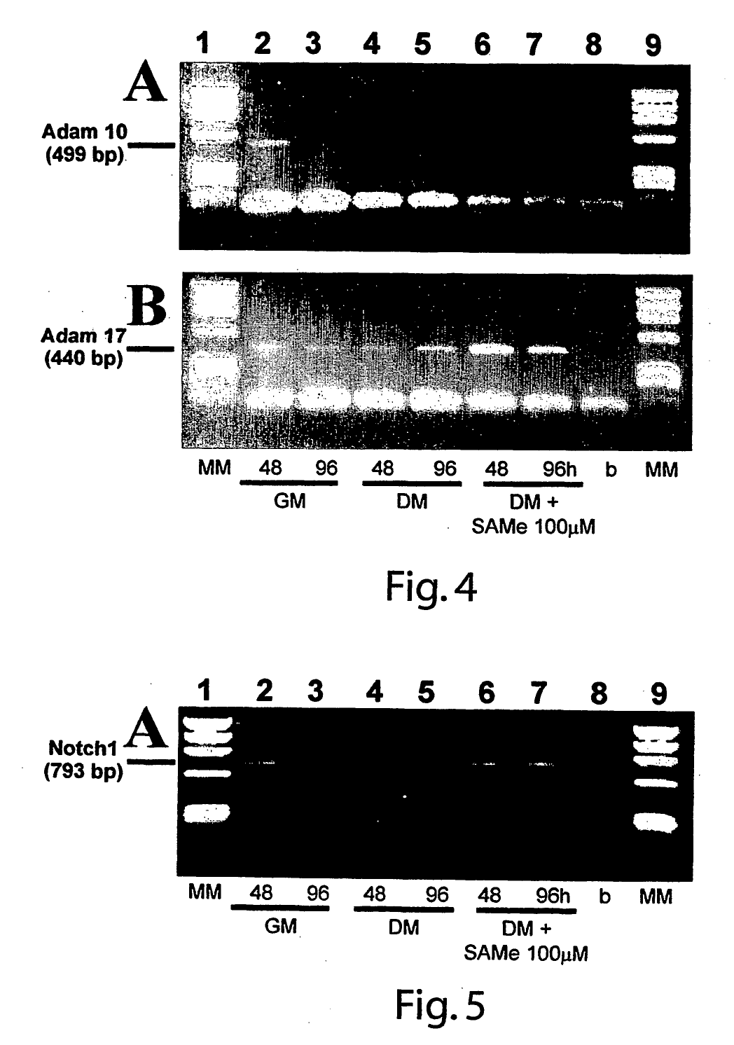

FIG. 4 shows the expression of Adam 10 (A) and Adam 17 (B) genes after 48 and 96 hours of culture. Lanes 4 and 5: DM; lanes 2 and 3: GM; lanes 6 and 7: SAM in DM.

FIG. 5 shows the expression of Notch1 gene after 48 and 96 hours of culture. Lanes 2 and 3: GM; lanes 4 and 5: DM; lanes 6 and 7: SAM in DM.

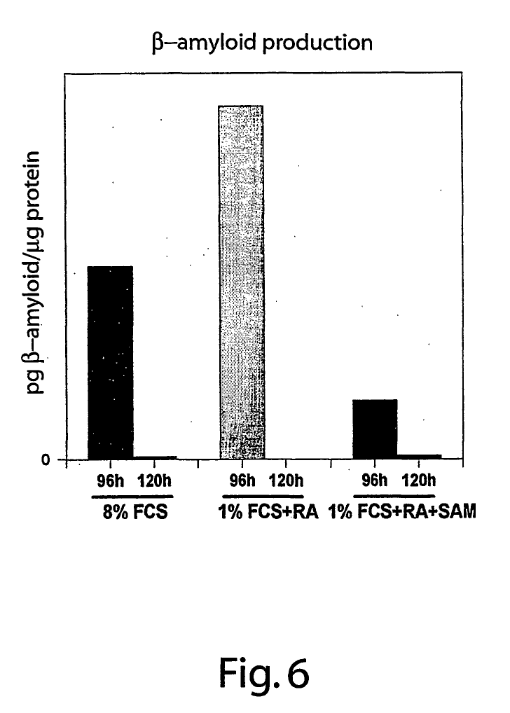

FIG. 6 shows the plot of the 1-40 β-amyloid peptide production after 96 and 120 hours.

### EXAMPLE 1

### Study About the Effects of S-Adenosylmethionine Disulfonate Administration on the Expression of Presenilin-1, Presenilin-2 and β-Secretase Genes in Human Neuroblastoma SK-N-SH Cells

By RT-PCR experiments (DNA polymerase chain reaction after reverse transcriptase reaction) it was showed a remarkable repression of the presenilin-1 gene expression, along with the block of the expression increase of the β-amyloid protein precursor, β-secretase and presenilin-2 following the administration of S-adenosylmethionine disulfonate into the cell culture. Furthermore it was demonstrated, by HPLC experiments, the actual S-adenosylmethionine passage into the cells. Values measured in SAM treated cell lysate are 6 fold higher than endogenous SAM. Protein Western Blot experiments showed a dramatic decrease of the presenilin-1 protein synthesis in the S-adenosylmethionine disulfonate treated cells (FIG. 3).

Methods

Cell Culture

SK-N-SH human neuroblastoma cell line was cultured, respectively, in HAM F14 (14) medium supplemented with 8-% foetal calf serum (GM), in F14 medium supplemented with 1% foetal calf serum and 10 μM retinopic acid (DM) and in DM, in the presence of 100 μM SAM. The cultures were re-fed every second day with the appropriate medium. The times indicate-are referred-to medium change as O-day.

HPLC Assays

Cell cultures were rinsed twice with phosphate buffered saline and frozen at −80° C. After thawing cells were scraped into 1 ml of deionized water and sonified for 15 seconds in ice. The macromolecules were precipitated from 1.5 M PCA at 4° C. for 1 hour adjusting the pH at 4-5 with KOH and then centrifuged for 15 minutes at 9000×g. The supernatants were freeze-dried. The HPLC measurements were carried out using a Varian HPLC System. The samples were dissolved in water and injected onto reverse-phase column (C-18) using a water-acetonitrile mobile-phase.

RNA Extraction and Expression Assay

Total RNA extraction was carried out using the acidified phenol procedure. For the expression studies by RT-PCR a reverse transcription was performed with 1 μg of total RNA using 50 pmol of oligo-d(T>)₁₆ with 50 units of M-MuLV reverse transcriptase at 42° C. for one hour, followed by heat inactivation at 94° C. for 5 minutes. Total reaction volume in the assay buffer was 20 μl, as suggested by the manufacturer. The subsequent amplification reactions were carried out for 20 to 30 cycles (1′ at 94° C., 1′ at annealing temperature, 1′30″ at 72° C.). The use of β-actin as internal standard allowed to control the processing of equal amount samples.

DNA Extraction and Methylation Assays by Multiplex-HpaII/PCR

Genomic DNA was extracted using a standard phenol/chloroform method followed by ethanol precipitation. Genomic DNA was treated with both of the following restriction endonucleases: i) EcoRI, which has no recognition sites internal to the amplified fragments; ii)-HpaII, which has a recognition site internal to-the amplified region and is methylation sensitive (i.e. it fails to cut if the CCGG recognition sequence is methylated at any C). 1.5 μg of genomic DNA were digested overnight at 37° C. with 5 units of enzyme and then with 3 units more for additional 6 hours, in a final volume of 40 μl of the buffer provided by the manufacturer. The subsequent amplification reactions were carried cut for 30 to 40 cycles (1′ at 94° C., 1′ at annealing temperature, 4′30″ at 72° C.) (15).

Gel Electrophoresis and Analysis of PCR Product

Aliquots of the PCR products (15 μl) were examined by electrophoresis in 1.5% agarose gel. Each gel was scanned by a CCD camera and acquired on a computerised densitometer. The specificity of the fragments was assessed by restriction analysis.

Western Blot Analysis

Detergent lysates were prepared from SK-N-SH cells in the presence of protease inhibitors (leupeptin, pepstatin, PMSF, 5 μg/ml each); protein extracts were performed after 5 days of culture in either GM or DM or DM supplemented with 100 μM SAM.

10 μg of each protein extract were run on 8% PAGE for APP analysis and on 12% PAGE for PS1 analysis, and then blotted on nitrocellulose. APP was detected by a monoclonal antibody 22C11 (Boerhinger Mannheim) recognising three major bands at 116, 110, 106 KD. PS1 was detected by a monoclonal antibody (MAB1563 Chemicon) recognising a 31 KD band.

Results

In FIG. 1 the gene expression is reported: APP (A), β-secretase (B), PS1 (C) and PS2 (D), after 48 or 96 hours of culture and on the right the plots of the O.D. values.

In all investigated genes the expression increased at 96 hours both in GM and DM (lanes 3 and 5) and was slightly higher in DM than in GM. For all investigated genes the expression thereof seems to be repressed in the presence of SAM (lanes 6-7). SAM seems to accelerate the gene expression at 48 hours, while, at 96 hours APP, β-secretase and PS2 did not increase and resulted inhibited if compared to 96 hours without SAM. On the contrary PS1 resulted markedly down-regulated at 96 hours.

The analysis of methylated sites on the PS1 gene promoter (FIG. 2) shows clear hypomethylation at 72 hours in correspondence with an accentuated gene expression at 96 hours (FIG. 1), while it is clear as much the hypomethylation reduction at 72 hours in the presence of SAM (about 1:2 ratio) with consequent reduction of the PS1 expression at 96 hours (see panel B in FIG. 2 and panel C in FIG. 1).

The results of the Western Blot analysis are reported in FIG. 3 wherein panel A shows the three APP isoforms which did not change with cultural conditions and panel B shows the marked reduction of PS1 expression induced by SAM addition.

### EXAMPLE 2

### Study About the Effects of S-Adenosylmethionine Administration on the Expression of Adam 10 (16) and Adam 17 (17) (α-Secretase) Genes in SK-N-SH Human Neuroblastoma Cells

By the same technique as described for Example 1 it was showed that following the administration of S-adenosylmethionine the expression of α-secretase encoding genes not only is not reduced but, on the contrary, is increased in comparison to the cells grown in absence of the compound (FIG. 4)

Methods

See “Example 1”

Results

In FIG. 4 the expression of Adam 10 (A) and Adam 17 (B) genes after 48 and 96 hours of culture is showed.

Adam 10 expression is apparent only at 48 hours and in DM it is lower (lane 4) than the signal detectable in GM (lane 2). In the presence of SAM the gene expression seems to be the same as that detectable in DM.

Adam 17 expression, on the contrary, is detectable at both culture times under all experimental conditions and it is the same both in GM (lanes 2 e 3) and DM (lanes 4 and 5). In the presence of SAM it is clear a gene over-expression at both of the times being more remarkably at 48 hours.

It is therefore apparent that in the presence of SAM (lanes 6 and 7) not only PS1 expression (γ-secretase) is decreased but on the contrary the expression of at least either of α-secretases is increased, suggesting a shifting of the APP processing favouring the not amyloidogenic cutting.

### EXAMPLE 3

### Study About the Effects of S-Adenosylmethionine Administration on the Expression of Notch1 Gene in SK-N-SH Human Neuroblastoma Cells

By the same technique as described for Example 1 it was showed that following the administration of S-adenosylmethionine the expression of Notch1 encoding gene not only is not reduced, but on the contrary, is increased in comparison to the cells grown in absence of the compound (FIG. 5).

Methods

See “Example 1”

Results

In FIG. 5 the expression of Notch1 gene after 48 and 96 hours of culture is showed. The expression thereof is detectable at both culture times under all experimental conditions and it is decreased in GM at 96 hours in comparison to that at 48 hours (lanes 2 and 3). In DM (lanes 4 and 5) the expression is the same as that observed in GM at 96 hours. In the presence of SAM (lanes 6 and 7) the gene expression at both culture times is comparable to that observed in GM at 48 hours.

It can be therefore concluded that SAM, although with reduction of PS1 expression (Example 1), does not modify the Notch1 expression, essential gene for the maturation of stem cells.

### EXAMPLE 4

### Study About the Effects of S-Adenosylmethionine Administration on the Production of β-Amyloid Peptide in SK-N-SH Human Neuroblastoma Cells

Using an ELISA assay it was showed that following the administration of S-adenosylmethionine the production of O-amyloid peptide is reduced in comparison to cells grown without the compound (FIG. 6).

Methods

Cell Culture

See “Example 1”

ELISA Assay

Cell culture supernatants were collected and frozen after 96 and 120 hours from the beginning of the culture. Successively they were concentrated using Amicon micro-concentrators according to the manufacturer instructions. 100 μl of concentrated medium were used to perform the immunoassay employing, according to the manufacturer instructions, the Biosource International kit for the detection of 1-40 amyloid. The amount of β-amyloid was normalised to the concentration of the proteins extracted from the corresponding cell lysates.

Results

In FIG. 6 the plot of the 1-40 β-amyloid peptide production after 96 and 120 hours is showed. There is nearly no production at 120 hours probably due to shorter residence time of the cells in the same medium. In fact the supernatants collected after 96 and 120 hours contacted the cells over 48 and 24 hours, respectively. Anyway the β-amyloid production in DM is higher than in GM, while the presence of SAM in the medium reduces the same remarkably.

Therefore it is possible to conclude that SAM, by reducing the PS1 expression (Example 1), inhibits the amyloidogenic cut on APP.

### BIBLIOGRAPHY

1. De Strooper, B, Nature 2000. June. 8.; 405.(6787.):627, 629, 405, 627, 629 (2000).

2.De Strooper, B., Saftig, P., Craessaerts, K., et al. Nature 391, 387-390 (1998).

3. Wolfe, M. S., Xia, W., Ostaszewski, B. L., Diehl, T. S., Kimberly, W. T. & Selkoe, D. J. Nature 398, 513-517 (1999).

4. Li, Y. M., Xu, M., Lai, M. T. et al. Nature 2000 June. 8; 405.(6787): 689M94. 405, 689-694 (2000).

5. Brown, M. S., Ye, J., Rawson, R. B. & Goldstein, J. L., Cell 2000. February. 18; 100(4.):391-8. 100, 391-398 (2000).

6. Wong, P. C., Zheng, H., Chen, H., et al., Nature, 387, 288-292 (1997).

7. Shen, J., Bronson, R. T., Chen, D. F., Xia, W., Selkoe, D. J. & Tonegawa, S. Cell 89, 629-639 (1997).

8. Bottiglieri, T. & Hyland. K. Acta Neurol. Scand. Suppl. 154, 19-26-(1994).

9. Morrison, L. D., Smith, D. D. & Kish, S. J., J. Neurochem. 67,1328-1331 (1996).

10. Handler, M., Yang, X. & Shen, J. Development 2000; 127 (12):2593-2606. 127, 2593-2606 (2000).

11. Bergman, Y. & Mostoslavsky, R., Biol. Chem. 379, 401-407 (1998).

12. Cross, S. H. & Bird, A. P., Curr. Opin. Genet. Dev. 5, 309-314 (1995).

13. Siegfried, Z. & Cedar, H., Curr. Biol. 7, R305-R307 (1997).

14. Vogel Z. Et al., Proc. Natl. Acad. Sci. U.S.A. 69, 3180-3184, 1972.

15. Lucarelli M. et al., J. Biol. Chem., 276, 7500-7506, (2001).

16. Rosendhal, M. S. et al., J. Biol. Chem., 272, 24588-24593, (1997).

17. Hirohata, S. et al., Genomics 54,178-179, (1998).

1. Use of S-adenosylmethionine and derivatives thereof for the preparation of a medicament for the treatment and prevention of Alzheimer's disease.

2. Use according to claim 1, wherein the derivative of S-adenosylmethionine is S-adenosylmethionine disulphonate.

3. Use of S-adenosylmethionine and derivatives thereof for the preparation of a medicament for the regulation of the expression of genes selected from the group consisting of O-secretase, presenilin-1, presenilin-2, β-amyloid protein precursor

4. Use of S-adenosylmethionine and derivatives thereof according to claim 3, wherein the regulation of the gene expression is by means of the modulation of methylation of regulatory gene regions.
